(19) United States
(12) Patent Application Publication
(10) Pub. No.: US 2010/0010367 A1
(43) Pub. Date: Jan. 14, 2010

(54) System and methods for monitoring during anterior surgery

(76) Inventors: Kevin T. Foley, Germantown, TN (US); Bret A. Ferree, Cincinnati, OH (US); James Gharib, San Diego, CA (US)

Correspondence Address: NuVasive;c/o CPA Global, P.O. Box 52050, Minneapolis, MN, 55402, US

(21) Appl. No.: 11/794,650

(22) PCT Filed: Dec. 30, 2005
(86) PCT No.: PCT/US05/47576

§ 371 Date: Sep. 30, 2009

Related U.S. Application Data
(60) Provisional application No. 60/640,863, filed on Dec. 30, 2004.

(51) Int. Cl. A61B 5/0488

(52) U.S. Cl. 600/546

(57) ABSTRACT

The present invention involves a system and methods for nerve testing during anterior surgery, including but not limited to anterior disc replacement surgery, nucleus replacement, and interbody fusion.

### CROSS REFERENCE TO RELATED APPLICATIONS

The present invention is an International Patent Application and claims the benefit of priority from commonly owned and co-pending U.S. Provisional Patent Application Ser. No. 60/640,863, entitled “System and Methods for Monitoring During Anterior Surgery” and filed on Dec. 30, 2004, the entire contents of which is hereby expressly incorporated by reference into this disclosure as if set forth in its entirety herein. The present application also incorporates by reference the following co-pending and co-assigned patent applications in their entireties: U.S. patent application Ser. No. 10/967,668, entitled “Surgical Access System and Related Methods,” filed on Oct. 18, 2004, PCT App. Ser. No. PCT/US2004/025550, entitled “System and Methods for Performing Dynamic Pedicle Integrity Assessments,” filed on Aug. 5, 2004.

### BACKGROUND OF THE INVENTION

I. Field of the Invention

The present invention relates generally to a system and methods aimed at surgery, and more particularly to system and methods for nerve testing during anterior surgery, including but not limited to anterior disc replacement surgery, nucleus replacement, and interbody fusion.

II. Discussion of the Prior Art

Anterior access to the lumbar spine may be obtained using one of a trans-peritoneal, retroperitoneal, or minimally invasive laparoscopic approach. Approaching the lumbar spine from an anterior direction has several potential advantages. Exposing the front of the spine, as opposed to the side or the back, generally allows for greater exposure and a more complete excision of the damaged disc. The anterior approach accesses the spine through the abdomen. Since the abdominal muscles can be retracted to the side and out of the way without being cut, anterior spinal access may create less morbidity for the patient. Despite the advantages anterior lumbar surgery offers, these anterior approaches (especially the trans-peritoneal and minimally invasive laparoscopic techniques), have experienced a decline in popularity. This decline is due, in part, to complications based on the presence of the hypogastric plexus, a complex of nerves which lies just in front of the lumbar spine. The hypogastric plexus innervates muscles in the pelvic region, including the bladder and anal sphincter muscles. The possibility of irreversibly damaging the hypogastric plexus when surgically exposing the anterior lumbar spine is a definite risk of anterior lumbar surgery. This can occur through inadvertent contact with a surgical accessory (dissector, knife blade, electrocautery tip, etc.) or while retracting the plexus out of the surgical access corridor. Such damage can inhibit the bladder sphincter from functioning properly. Loss of bladder sphincter function may result in retrograde ejaculation in men and possibly leave the individual sterile. This is especially true for trans-peritoneal and minimally invasive laparoscopic approaches, which tend to result in a much higher incidence of retrograde ejaculation than the retroperitoneal approach.

To help prevent such damage and better realize the possible advantages of an anterior approach to the lumbar spine, surgeons need a way to detect and monitor the hypogastric plexus during the procedure. The present invention is directed at addressing this previously unmet need.

### SUMMARY OF THE INVENTION

The present invention includes a system and related methods for determining the proximity and pathology of the hypogastric plexus in relation to surgical instruments employed in accessing the anterior lumbar spine.

According to a broad aspect, the present invention includes a surgical system, comprising a control unit and a surgical instrument. The control unit has at least one of computer programming software, firmware and hardware capable of delivering a stimulation signal, receiving and processing neuromuscular responses due to the stimulation signal, and identifying a relationship between the neuromuscular response and the stimulation signal. The surgical instrument has at least one stimulation electrode electrically coupled to the control unit for transmitting a stimulation signal. The control unit is capable of determining at least one of nerve proximity and nerve pathology for the hypogastric plexus, based on the identified relationship between a stimulation signal and a corresponding neuromuscular response.

In a further embodiment of the surgical system of the present invention, the control unit is further equipped to communicate at least one of alphanumeric and graphical information to a user regarding at least one of nerve proximity and nerve pathology of the hypogastric plexus.

In a further embodiment of the surgical system of the present invention, the hardware employed by the control unit to monitor neuromuscular response may comprise at least one of EMG electrodes or pressure sensors.

In a further embodiment of the surgical system of the present invention, the hardware employed by the control unit to monitor neuromuscular response comprises an EMG electrode positioned on a urinary catheter for monitoring bladder sphincter activity.

In a further embodiment of the surgical system of the present invention, the hardware employed by the control unit to monitor neuromuscular response comprises an EMG electrode contained on a device capable of insertion into the rectum for monitoring anal sphincter activity.

In a further embodiment of the surgical system of the present invention, the hardware employed by the control unit to monitor neuromuscular response comprises a pressure sensor positioned on a urinary catheter for monitoring bladder sphincter activity.

In a further embodiment of the surgical system of the present invention, the surgical instrument may comprise at least one of a device for providing a stimulation signal, a device for accessing the anterior lumbar spine, and a device for maintaining contact with the hypogastric plexus during surgery.

In a further embodiment of the surgical system of the present invention, the surgical instrument comprises a dilating instrument and the control unit determines the proximity between the hypogastric plexus and the instrument based on the identified relationship between the neuromuscular response and the stimulation signal.

In a further embodiment of the surgical system of the present invention, the surgical instrument comprises a tissue retractor assembly and the control unit determines the proximity between the hypogastric plexus and the instrument based on the identified relationship between the neuromuscular response and the stimulation signal.

In a further embodiment of the surgical system of the present invention, the surgical instrument comprises a nerve root retractor and the control unit determines nerve pathology based on the identified relationship between the neuromuscular response and the stimulation signal.

### BRIEF DESCRIPTION OF THE DRAWINGS

Many advantages of the present invention will be apparent to those skilled in the art with a reading of this specification in conjunction with the attached drawings, wherein like reference numerals are applied to like elements and wherein:

FIG. 1 is a perspective view of an exemplary surgical system 10 capable of nerve testing during anterior surgery;

FIG. 2 is a block diagram of the surgical system 10 shown in FIG. 1;

FIG. 3 is an illustration of a fingertip stimulator for delivering a stimulation current to nearby nerves during a surgical procedure;

FIG. 4 is a perspective view of a ring EMG electrode for monitoring EMG responses of the bladder sphincter;

FIG. 5 is an illustration showing the ring EMG electrode of FIG. 4 positioned on a urinary catheter for insertion to the bladder sphincter;

FIG. 6 is a side view of a probe device containing an EMG electrode for measuring EMG responses of the anal sphincter;

FIG. 7 is an illustration showing a microchip pressure sensor positioned on a urinary catheter for insertion to the bladder sphincter;

FIG. 8 is a graph illustrating a plot of the neuromuscular response (EMG) of a given myotome over time based on a current stimulation pulse (similar to that shown in FIG. 9) applied to a nerve bundle coupled to the given myotome;

FIG. 9 is a graph illustrating a plot of a stimulation current pulse capable of producing a neuromuscular response (EMG) of the type shown in FIG. 8;

FIG. 10 is an illustration (graphical and schematic) of a method of automatically determining the maximum frequency (F_{Max}) of the stimulation current pulses according to one embodiment of the present invention;

FIG. 11 is a graph illustrating a plot of peak-to-peak voltage (Vpp) for each given stimulation current level (I_{Stim}) forming a stimulation current pulse train according to the present invention (otherwise known as a “recruitment curve”);

FIGS. 12A-12D are graphs illustrating a rapid current threshold-hunting algorithm according to one embodiment of the present invention;

FIG. 13 is a series of graphs illustrating a multi-channel rapid current threshold-hunting algorithm according to one embodiment of the present invention;

FIG. 14 is an exemplary screen display illustrating one embodiment of the nerve proximity (detection) function of the present invention; and

FIG. 15 is a graph illustrating recruitment curves for a generally healthy nerve (denoted “A”) and a generally unhealthy nerve (denoted “B”) according to the nerve pathology determination method of the present invention.

### DESCRIPTION OF THE PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time consuming, but would nevertheless be a routine undertaking for those of ordinary skill in the art having the benefit of this disclosure. The systems disclosed herein boast a variety of inventive features and components that warrant patent protection, both individually and in combination.

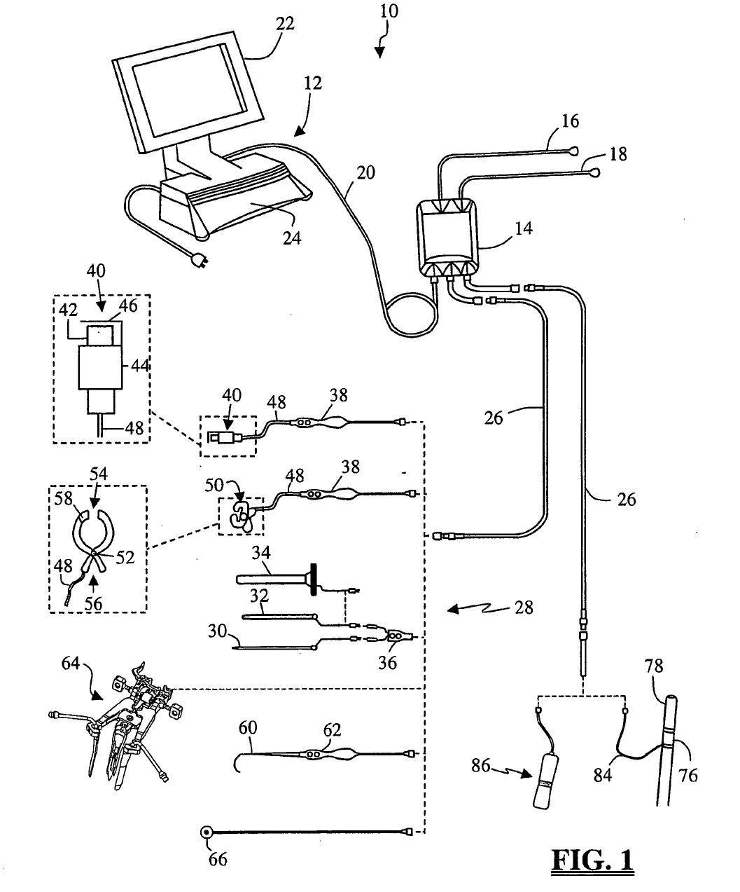

The present invention is directed at nerve testing before, during, and/or after anterior lumbar surgery, including but not limited to total disc replacement, nucleus replacement, and interbody fusion surgeries. The invention provides nerve related information to help surgeons avoid damaging the nerves lying in front of the lumbar spine. FIG. 1 illustrates, by way of example only, a surgical system 10 capable of carrying out nerve testing functions including, but not necessarily limited to nerve proximity testing and nerve pathology monitoring. In an exemplary embodiment the surgical system 10 carries out nerve testing functions particularly on the hypogastric plexus.

The surgical system 10 includes a control unit 12, a patient module 14, a muscle activity sensor (such as EMG electrodes 76, 88, or pressure sensor 94) coupled to the patient module 14, an anode electrode 18 providing a return path for the stimulation current, a common electrode 16 providing a ground reference to pre-amplifiers in the patient module 14, and a host of surgical accessories 28 capable of being coupled to the patient module 14 via one or more accessory cables 26. The surgical accessories 28 may include, but are not necessarily limited to, stimulation accessories including (but not limited to) a finger tip electrode 68, surgical access components (such as a K-wire 30, one or more dilating cannula 32, a working cannula 34, tissue retraction assembly 64) and neural pathology monitoring devices (such as a nerve root retractor 60). Although not shown, such surgical accessories may include (but are not limited to) an electrocautery device.

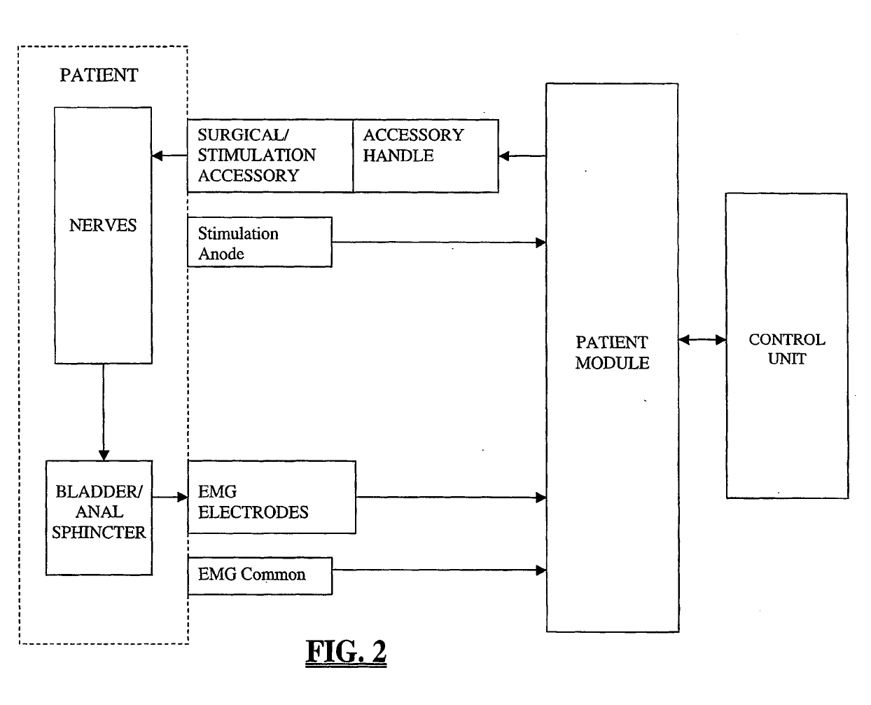

A block diagram of the surgical system 10 is shown in FIG. 2, the operation of which is readily apparent in view of the following description. The control unit 12 includes a touch screen display 22 and a base 24, which collectively contain the essential processing capabilities for controlling the surgical system 10. The touch screen display 22 is preferably equipped with a graphical user interface (GUI) capable of communicating information to the user and receiving instructions from the user. The base 24 contains computer hardware and software that commands the stimulation sources, receives digitized signals and other information from the patient module 14, processes the neuromuscular responses, and displays the processed data to the operator via the display 22. The primary functions of the software within the control unit 12 include receiving user commands via the touch screen display 22, activating stimulation in the requested mode (such as nerve proximity or nerve pathology), processing signal data according to defined algorithms (described below), displaying received parameters and processed data, and monitoring system status.

The patient module 14 is connected via a data cable 20 to the control unit 12, and contains the electrical connections to all electrodes, signal conditioning circuitry, stimulator drive and steering circuitry, and a digital communications interface to the control unit 12. In use, the control unit 12 is situated outside but close to the surgical field (such as on a cart adjacent the operating table) such that the display 22 is directed towards the surgeon for easy visualization. The patient module 14 should be located between the patient's legs, or may be affixed to the end of the operating table at mid-leg level using a bedrail clamp. The position selected should be such that all neuromuscular sensors can reach their farthest desired location without tension during the surgical procedure.

In a significant aspect of the present invention, the information displayed to the user on the display 22 may include, but is not necessarily limited to, alpha-numeric and/or graphical information regarding nerve proximity, nerve pathology, myotome/EMG levels, pressure levels, stimulation levels, advance or hold instructions, the instrument in use, and the EMG device in use. In one embodiment (set forth by way of example only) the display includes the following components as set forth in Table 1:

TABLE 1

| Screen Component | Description |
|---|---|
| Spine Image 106 | An image of the human body/skeleton showing the electrode |
| | placement on or within the body, with labeled channel |
| | number tabs on each side (1-4 on the left and right). Left and |
| | right labels will show the patient orientation. The Channel |
| | number tabs may be highlighted or colored depending on the |
| | specific function being performed and the specific electrodes |
| | in use. |
| Display Area 116 | Shows procedure-specific information including stimulation |
| | results 102. |
| Myotome 108 & | A label to indicate the Myotome name and corresponding |
| Nerve 110 Names | Nerve(s) associated with the channel of interest. |
| Advance/Hold 104 | In one embodiment, when in Detection mode, an indication |
| | of “Advance” will show when it is safe to move the surgical |
| | accessory forward (such as when the minimum stimulation |
| | current threshold Iₜₕᵣₑₛₕ (described below) is greater than a |
| | predetermined value, indicating a safe distance to the nerve) |
| | and “Hold” is displayed when it is unsafe to advance the |
| | surgical accessory (such as when the minimum stimulation |
| | current threshold Iₜₕᵣₑₛₕ (described below) is less than a |
| | predetermined value, indicating that the nerve is relatively |
| | close to the accessory) and during proximity calculations. |
| Color Indication | Enhances stimulation results with a color display of green, |
| | yellow, or red corresponding to the relative safety level |
| | determined by the system. |
| Function | Graphics and/or name to indicate the currently active |
| Indicator 98 | function (Detection, Nerve Retractor). In an alternate |
| | embodiment, Graphics and/or name may also be displayed to |
| | indicate the instrument in use, such as the Finger Tip |
| | Electrode, K-wire, Dilating Cannula, Working Cannula, |
| | Retractor Assembly, and/or Nerve Root Retractor, and |
| | associated size information, if applicable, of the cannula, |
| | with the numeric size. If no instrument is in use, then no |
| | indicator is displayed. |
| Stimulation Bar 114 | A graphical stimulation indicator depicting the present |
| | stimulation status (ie . . . on or off and stimulation current level) |
| EMG waveforms | EMG waveforms may be optionally displayed on screen |
| | along with the stimulation results. |

The surgical system 10 accomplishes safe and reproducible access to the spine during anterior lumbar surgeries, including but not necessarily limited to total disc replacement, nucleus replacement, and interbody fusion. The surgical system 10 does so by electrically stimulating the hypogastric plexus while monitoring the corresponding myotome response of a muscle or muscles (preferably the bladder sphincter) innervated by the hypogastric plexus. Monitoring may be conducted before, during, and after the establishment of an operative corridor, through the abdominal area, to the surgical target site in the anterior spine. Analysis of the muscle response may provide the surgeon with information relating to at least one of proximity and pathology of the hypogastric plexus. Stimulation may be achieved via one or more stimulation electrodes 66 positioned on a stimulation accessory, stimulation electrodes at the distal end of the surgical access components 30-34, or on a tissue retraction assembly 64. Additionally, non-evoked muscle activity may be monitored via free running EMG to provide additional information on stretching of the hypogastric plexus, as well as nerve and bladder function post-operatively. Free running EMG waveforms may be shown on the display screen 22 at the option of the user.

The surgical access components 30-34 are designed to bluntly dissect the tissue between the patient's skin and the surgical target site. Prior to this, due to the anterior approach, a general surgeon or access surgeon will first undertake to either move the peritoneum and the organs contained within it aside (i.e. retroperitoneal approach) or create a passageway through the peritoneum to the spine (i.e. trans-peritoneal and minimally invasive laparoscopic approaches) to allow the introduction of the access system of the present invention. An initial dilating cannula 32 is advanced towards the target site, preferably after having been aligned using any number of commercially available surgical guide frames. An obturator (not shown) may be included inside the initial dilator 32 and may similarly be equipped with one or more stimulating electrodes. Once the proper location is achieved, the obturator (not shown) may be removed and the K-wire 30 inserted down the center of the initial dilating cannula 32 and docked to the given surgical target site, such as the annulus of an intervertebral disc. Cannulae of increasing diameter are then guided over the previously installed cannula 32 until the desired lumen is installed. By way of example only, the dilating cannulae 32 may range in diameter from 6 mm to 30 mm. The working cannula 34 is installed over the last dilating cannula 32 and then all the dilating cannulae 32 are removed from inside the inner lumen of the working cannula 34 to establish the operative corridor therethrough. In a preferred embodiment the access components are coupled to the surgical system 10 using an electrical coupling device 40 such as that described below. Alternatively, a stimulator driver 36 is provided to electrically couple the particular surgical access component 30-34 to the patient module 14 (via accessory cable 26). In a preferred embodiment, the stimulator driver 36 includes one or more buttons for selectively activating the stimulation current and/or directing it to a particular surgical access component.

Additional and/or alternative surgical access components such as, by way of example only, a tissue retraction assembly 64 (FIG. 1) may be coupled to the system 10 and employed to provide safe and reproducible access to a surgical target site. Tissue retraction assembly 64 and various embodiments and uses thereof have been shown and described in the above referenced co-pending and commonly assigned U.S. patent application Ser. No. 10/967,668, entitled “Surgical Access System and Related Methods,” filed on Oct. 18, 2004, the entire contents of which are expressly incorporated by reference as if set forth herein in their entirety.

In yet another alternative, a stimulation accessory may be used in conjunction with traditional surgical access tools to provide safe access to the anterior target site. Traditional surgical tools may be employed to create an operating corridor to the anterior lumbar spine while a stimulation accessory is simultaneously employed to detect the nearby hypogastric plexus. The stimulation accessory may be embodied in any number of suitable forms that can safely advance a stimulation electrode 66, through the access corridor and into contact with the surrounding tissue. By way of example only, the stimulation accessory may simply comprise a blunt probe fashioned with a stimulation electrode 66 on the blunt end. By way of further example, any of a variety of electrocautery devices used to stop bleeding during surgery may be advantageously fashioned with a stimulation electrode 66 according to the present invention. Additionally, the stimulation accessory may comprise a fingertip stimulator 68 as shown in FIG. 3. A small stimulation electrode 66 may be situated in the fingertip region of a surgical glove 70. The electrode 66 may be adhered to a standard surgical glove with a biocompatible adhesive or the electrode may be manufactured into a specially designed surgical glove. Lead wires 74 extending from the electrode 66 and connecting to an accessory cable 26 may be adhered or attached along the glove and arm so as not to interfere with the-surgeon's movements during the procedure. Stimulator driver 36 or an accessory handle 38 electrically couple the stimulation accessory to the patient module 14 (via accessory cable 26) and preferably include one or more buttons for selectively activating the stimulation current.

Alternatively, an electric coupling device 40 may be attached to stimulation accessory handle 38. The electric coupling device 40 may be utilized to couple traditional surgical tools, such as (by way of example only) an electrocautery device, to the surgical system 10. In this manner, a stimulation signal may be passed directly through traditional surgical tools while the tool is in use.

The electric coupling device 40 may comprise a number of possible embodiments which permit the device to attach and hold a surgical tool while allowing transmission of a stimulation signal to the tool. One such electric coupling device 40 utilizes a spring-loaded plunger to hold the surgical tool and transmit the stimulation signal. The plunger 42 is composed of a conductive material such as metal. A nonconductive housing 44 partially encases the plunger 42 about its center. Extending from the housing 44 is an end plate 46. An electrical cable 48 connects the electric coupling device 42 to the handle 38. A spring (not shown) is disposed within the housing 44 such that in a natural or “closed” state the plunger 42 is situated in close proximity to the endplate 46. Exerting a compressive force on the spring (such as by pulling the cable 48 while holding the housing 44) causes a gap between the end plate 46 and the plunger 42 to widen to an “open” position, thereby allowing insertion of a surgical tool between the end plate 46 and plunger 42. Releasing the cable 48 allows the spring to return to a “closed” position, causing the plunger 42 to move laterally back towards the endplate such that a force is exerted upon the surgical tool and thereby holds it in place between the endplate 46 and the plunger 42. Thereafter the electrical stimulus may be passed from the handle 38 through the cable 48 and plunger 42 to the surgical tool.

Alternatively, the electrical coupling device may be embodied in the form of a clip 50. The clip 50 is comprised of two prongs hingedly coupled at a coupling point 52 such that the clip 50 includes an attachment end 54 and a non-attachment end 56. A stimulation electrode 58 is disposed on the attachment end 54 and communicates with an electric cable 48 extending from the non-attachment end 56 to the handle 38. In a “closed” position the prong ends at the attachment end 54 touch. Depressing the prongs at the non-attachment end 56 in a direction towards each other causes a gap to form between the prong ends at the attachment end 54. Positioning the “opened” attachment end 54 over a desired surgical tool and releasing the force on the non-attachment end 56 causes the attachment end 54 to pinch tight on the surgical tool and thereby allow the electrical stimulus to pass from the stimulation accessory handle 38, through the stimulation electrode 58, to the surgical tool.

The surgical system 10 accomplishes neural pathology monitoring during anterior lumbar surgery by electrically stimulating the retracted hypogastric plexus via one or more stimulation electrodes at the distal end of the nerve retractor 60 while monitoring the neuromuscular responses of a muscle group innervated by the hypogastric plexus. Analysis of the responses may then be used to assess the degree to which retraction of the nerve or neural structure affects the nerve function over time, as will be described with greater particularity below. One advantage of such monitoring, by way of example only, is that the conduction of the nerve may be monitored during the procedure to determine whether the neurophysiology and/or function of the nerve changes as the result of the retraction. The nerve retractor 60 may comprise any number of suitable devices capable of maintaining contact with the hypogastric plexus. The nerve retractor 60 may be dimensioned in any number of different fashions, including having a generally curved distal region (shown as a side view in FIG. 1 to illustrate the concave region where the nerve will be positioned while retracted), and of sufficient dimension (width and/or length) and rigidity to maintain the retracted nerve in a desired position during surgery. The nerve retractor 60 may also be equipped with a handle 62 having one or more buttons for selectively applying the electrical stimulation to the stimulation electrode(s) at the end of the nerve retractor 60. In one embodiment, the nerve retractor 60 is disposable and the handle 62 is reusable and autoclavable.

In a preferred embodiment neuromuscular response monitoring is conducted via EMG. Monitoring of EMG responses corresponding to hypogastric plexus stimulation is preferably accomplished via an EMG electrode placed in contact with the bladder sphincter located at the urethra-bladder junction or bladder neck. The EMG responses provide a quantitative measure of the nerve depolarization caused by the electrical stimulus. Analysis of the EMG responses in relation to the stimulation electrode is then used to determine the proximity or pathology of the hypogastric plexus as will be described with particularity below.

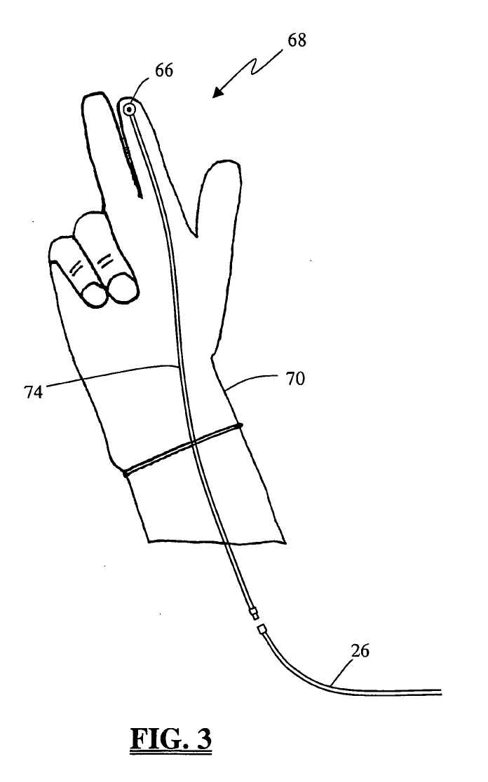

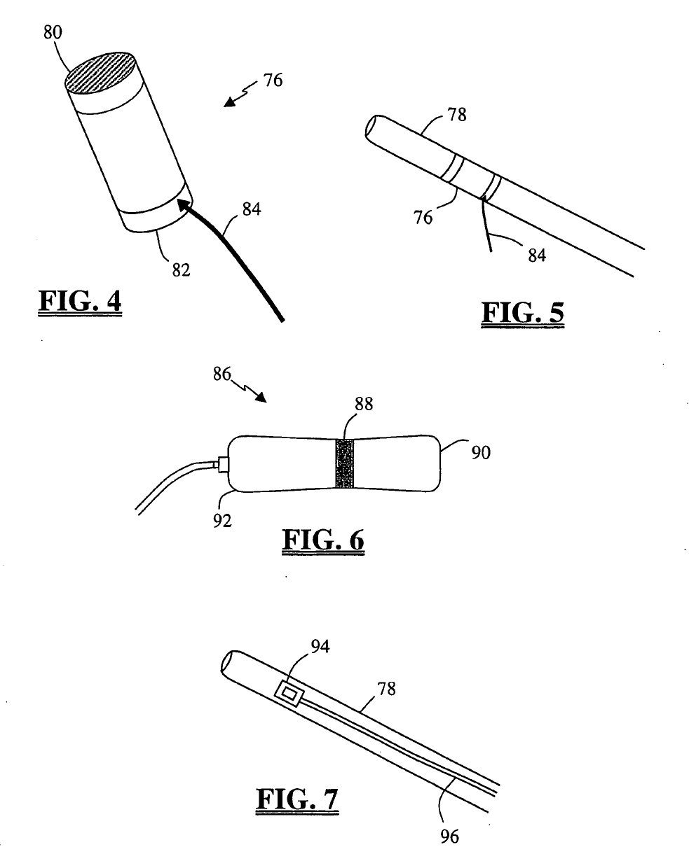

FIG. 5 illustrates a preferred method for deploying an EMG electrode to monitor bladder sphincter activity. An EMG electrode 76 is affixed to a position near the insertion end of a urinary catheter 78 such that when the 78 is inserted into the bladder, the electrode 76 is placed in contact with the sphincter muscle. One way to accomplish this is through the use of a ring electrode 76 shown in FIG. 4. The ring electrode 76 has a generally elongated annular shape defined by a distal end 80 and a proximal end 82. Lead wires 84 attached to the proximal end 82 communicatively link the electrode 76, via accessory cable 26, to the patient module 14. As pictured in FIG. 5 the electrode 76 may be mounted on the surface of catheter 78 by passing the catheter through the center of electrode 76 until a desired location on the catheter is reached. Electrode 76 may be fixed in position on the catheter by a number of suitable means including, but not-necessarily limited to, a biocompatible adhesive, providing a slit in electrode 76 extending from distal end 80 to proximal end 82 and thereafter crimping the electrode on the desired location, and providing a ring electrode 76 with a tapered circumference corresponding to an opposing taper along the insertion end of catheter 78, thereby securing an interference fit at a specific point on the catheter. Alternatively, the ring electrode 76 may be fixed to any device, other than the urinary catheter 78, which is capable of passing through the urethra to the bladder. It is also contemplated that a urinary catheter may be specifically designed and manufactured to contain a fully integrated EMG electrode. Although not shown, it will be appreciated that a variety of other electrodes may be employed to measure the EMG response of the bladder sphincter. By way of example only, a fine wire EMG electrode may be inserted into the bladder sphincter either percutaneously or via the urethra. A needle electrode may also be inserted into the bladder sphincter.

In an alternate embodiment, EMG monitoring may be conducted on the anal sphincter which is also innervated by the hypogastric plexus. A variety of EMG electrodes may be employed to monitor anal sphincter activity. By way of example only, FIG. 6 illustrates a probe device 86 containing an EMG electrode 88 for insertion into the rectum. The probe device 86 has an internal end 90 and an external end 92 with a recording electrode located therebetween. Internal end 90 is inserted through the anal sphincter until electrode 88 comes into contact with the anal sphincter. Alternatively, surface electrodes may be placed around the anal sphincter. In still another preferred embodiment, the system 10 employs both bladder sphincter and anal sphincter monitoring simultaneously via multiple EMG channels.

In yet another embodiment, the surgical system 10 may employ pressure sensors (as opposed to EMG electrodes), communicatively linked to the system, to monitor muscle activity of the bladder and anal sphincters. A preferred method of deploying a pressure sensor to the bladder sphincter is to couple a sensor to the insertion end of a urinary catheter such that the sphincter may contract around the sensor when the catheter is inserted into the bladder. By way of example only, FIG. 7 shows a pressure sensing microchip 94, communicatively linked to the patient module via lead wires 96, adhered to the outside surface of urinary catheter 78. Stimulation of the hypogastric plexus causes the bladder sphincter to close around sensor creating a detectable pressure increase which is measured by the system. Pressure increase may provide a quantitative measure of the nerve depolarization caused by the electrical stimulus. Analysis of the pressure increase in relation to the stimulation electrode may then be used to determine at least one of proximity, direction, or pathology of the hypogastric plexus.

In some cases, when a nerve is compressed or stretched, it will emit a burst or train of spontaneous nerve activity. The system 10 may conduct free running EMG (and/or pressure sensing) on the bladder and/or anal sphincter to capture this activity. Spontaneous EMG activity from the bladder and/or anal sphincters may alert the surgeon to over-stretching of the hypogastric plexus during retraction of the nerve, this is particularly useful when pathology monitoring of the nerve is not being conducted. An audio pick-up (not shown) may also be provided as an optional feature according to the present invention. The audio pick-up is capable of transmitting sounds representative of such activity such that the surgeon can monitor this response on audio to help him determine if there has been stress to one of the nerves.

Free running EMG may also be performed to monitor the post-operative condition of the patient. Spontaneous contractions of the bladder sphincter or other muscles after surgery may alert the surgeon to potential complications which could require further attention, such as (by way of example only) nerve injury caused by an epidural hematoma. Additionally, post-operative free run monitoring performed on the lower extremities may be beneficial to the patient and is provided for by the surgical system 10. To accomplish this, one or more EMG electrodes may be connected to the system 10 and placed on the skin over the major muscle groups of the legs. In one embodiment, an EMG harness (not shown) is provided having 8 pairs of EMG electrodes (4 per side) and may be positioned over the legs, as shown by way of example only, in Table 2 below:

| Myotome | Nerve | Spinal Level |
|---|---|---|
| Right Vastus Medialis | Femoral | L2, L3, L4 |
| Right Tibialis Anterior | Peroneal | L4, L5 |
| Right Biceps Femoris | Sciatic | L5, S1, S2 |
| Right Gastroc. Medial | Post Tibialis | S1, S2 |
| Left Vastus Medialis | Femoral | L2, L3, L4 |
| Left Tibialis Anterior | Peroneal | L4, L5 |
| Left Biceps Femoris | Sciatic | L5, S1, S2 |
| Left Gastroc. Medial | Post Tibialis | S1, S2 |

It should be appreciated that any of a variety of electrodes can be employed to monitor the muscle groups of the lower extremities, including but not necessarily limited to surface pad electrodes and needle electrodes.

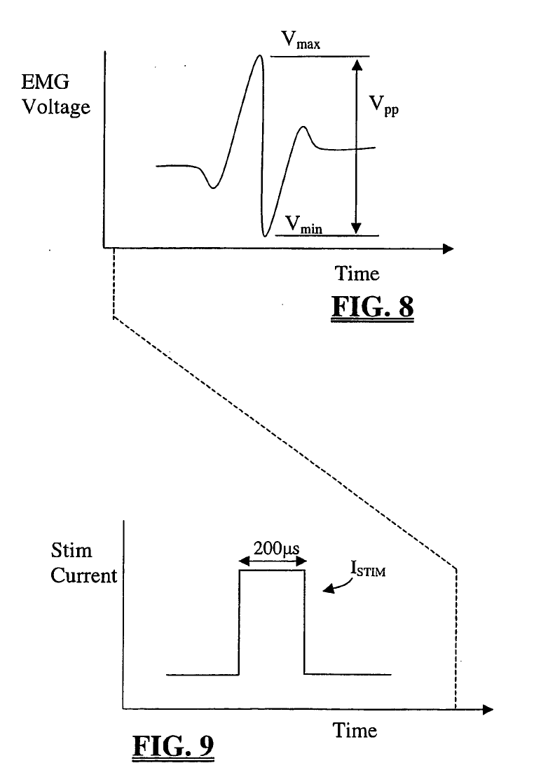

The nerve testing functions mentioned above (nerve proximity and nerve pathology) are based on assessing the evoked response of the various muscles myotomes monitored by the surgical system 10, via EMG electrodes 76 or 88. This is best shown in FIG. 8-9, wherein FIG. 8 illustrates the EMG of a monitored myotome to the stimulation current pulse shown in FIG. 9. The EMG response can be characterized by a peak-to-peak voltage of V_{PP}=Vₘₐₓ−Vₘᵢₙ. The stimulation current may be coupled in any suitable fashion (ie. AC or DC) and comprises monophasic pulses of 200 μs duration, with an amplitude and frequency that is controlled and adjusted by the software. For each nerve and myotome there is a characteristic delay from the stimulation current pulse to the EMG response (typically between 5 to 20 ms). To account for this, the frequency of the current pulses is set at a suitable level such as, in a preferred embodiment, 4 Hz to 10 Hz (and most preferably 4.5 Hz), so as to prevent stimulating the nerve before it has a chance to recover from depolarization.

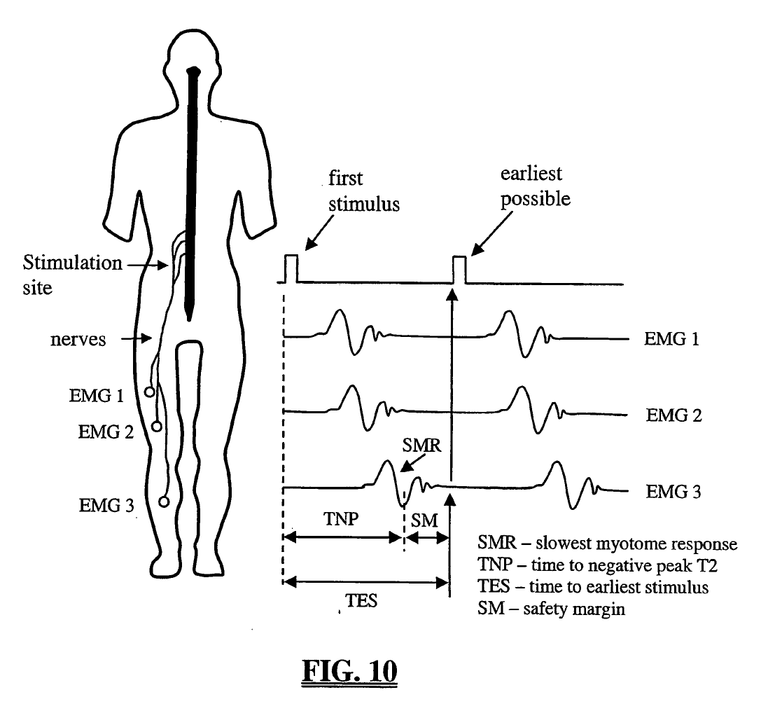

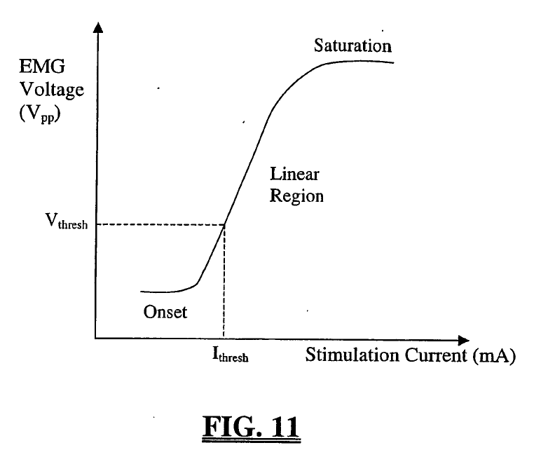

FIG. 10 illustrates an alternate manner of setting the maximum stimulation frequency (Fₘₐₓ), to the extent it is desired to do so rather than simply selecting a fixed maximum stimulation frequency (such as 4.5 Hz) as described above. According to this embodiment, the maximum frequency of the stimulation pulses is automatically adjusted. After each stimulation, Fₘₐₓ will be computed as: Fₘₐₓ=1/(T2+T_{Safety Margin}) for the largest value of T2 from each of the active EMG channels. In one embodiment, the Safety Margin is 5 ms, although it is contemplated that this could be varied according to any number of suitable durations. Before the specified number of stimulations, the stimulations will be performed at intervals of 100-120 ms during the bracketing state, intervals of 200-240 ms during the bisection state, and intervals of 400-480 ms during the monitoring state (bracketing, bisection and monitoring states are discussed in detail below). After the specified number of stimulations, the stimulations will be performed at the fastest interval practical (but no faster than Fₘₐₓ) during the bracketing state, the fastest interval practical (but no faster than Fₘₐₓ/2) during the bisection state, and the fastest interval practical (but no faster than Fₘₐₓ/4) during the monitoring state. The maximum frequency used until Fₘₐₓ is calculated is preferably 10 Hz, although slower stimulation frequencies may be used during some acquisition algorithms. The value of Fₘₐₓ used is periodically updated to ensure that it is still appropriate. For physiological reasons, the maximum frequency for stimulation will be set on a per-patient basis. Readings will be taken from all myotomes and the one with the slowest frequency (highest T2) will be recorded.

A basic premise behind the neurophysiology employed for nerve testing in the present invention is that each nerve has a characteristic threshold current level (I_{Thresh}) at which it will depolarize. Below this threshold, current stimulation will not evoke a significant neuromuscular response. Once the stimulation threshold (I_{Thresh}) is reached, the evoked response is reproducible and increases with increasing stimulation until saturation is reached as shown in FIG. 11. This is known as a “recruitment curve.” In one embodiment, a significant EMG response is defined to have a Vₚₚ of approximately 100 uV. The lowest stimulation current that evokes this threshold voltage (V_{Thresh}) is called I_{Thresh}. Iₜₕᵣₑₛₕ decreases as the degree of electrical communication between a stimulation impulse and a nerve increases. Thus, monitoring Iₜₕᵣₑₛₕ can provide the surgeon with useful information By way of example only, communication between a stimulation impulse and a nerve is affected by the distance between the stimulation electrode and the nerve and as the proximity between the nerve and electrode decreases the Iₜₕᵣₑₛₕ decreases. Thus Iₜₕᵣₑₛₕ may be employed to provide the surgeon with a relative indication of distance (proximity) between the stimulation electrode to the nerve.

In order to obtain Iₜₕᵣₑₛₕ and take advantage of the useful information it provides, the peak-to-peak voltage (Vₚₚ) of each EMG response corresponding a given stimulation current (I_{Stim}) must be identified. This is complicated by the existence of stimulation and/or noise artifacts which may create an erroneous Vₚₚ measurement of the electrically evoked EMG response. To overcome this challenge, the surgical system 10 of the present invention may employ any number of suitable artifact rejection techniques such as those shown and described in full in the above referenced co-pending and commonly assigned PCT App. Ser. No. PCT/US 2004/025550, entitled “System and Methods for Performing Dynamic Pedicle Integrity Assessments,” filed on Aug. 5, 2004.

Having measured each Vₚₚ EMG response the Vₚₚ information is analyzed relative to the stimulation current in order to determine a relationship between the nerve and the given stimulation element transmitting the stimulation current. More specifically, the present invention determines these relationships (between nerve and the stimulation element) by identifying the minimum stimulation current (I_{Thresh}) capable of resulting in a predetermined Vₚₚ EMG response. According to the present invention, the determination of I_{Thresh} may be accomplished via any of a variety of suitable algorithms or techniques.

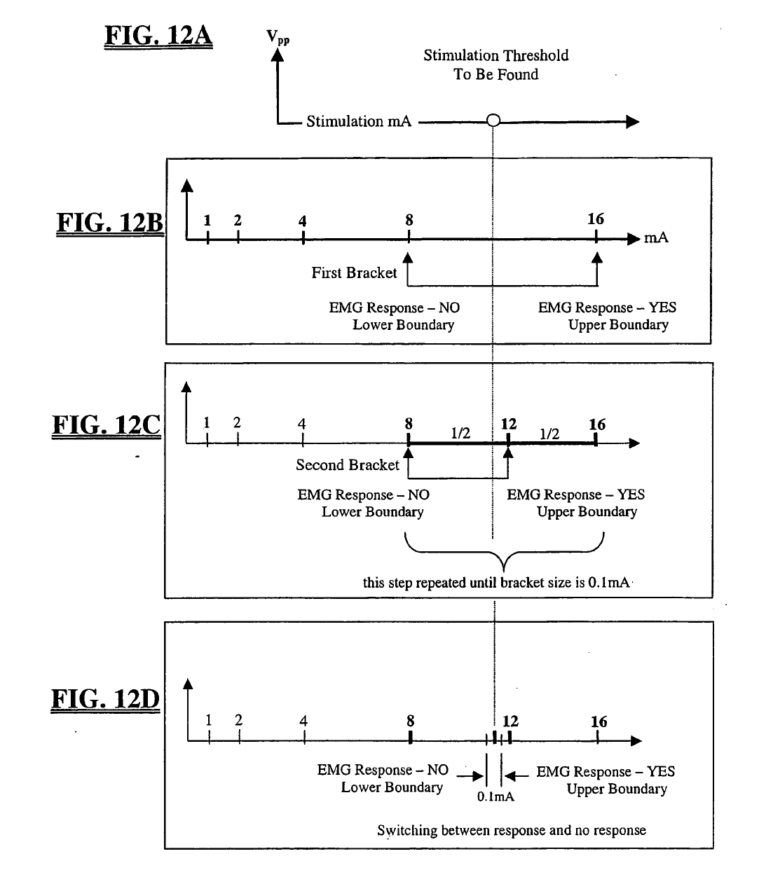

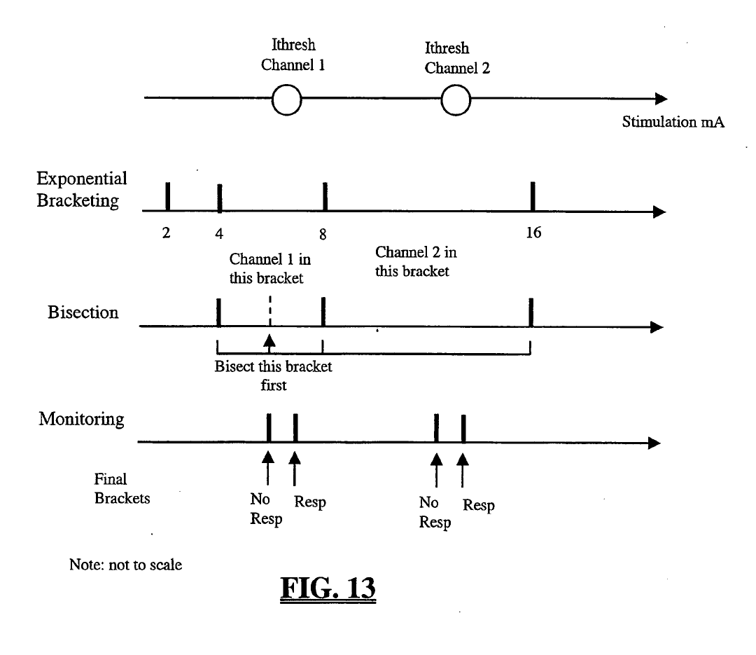

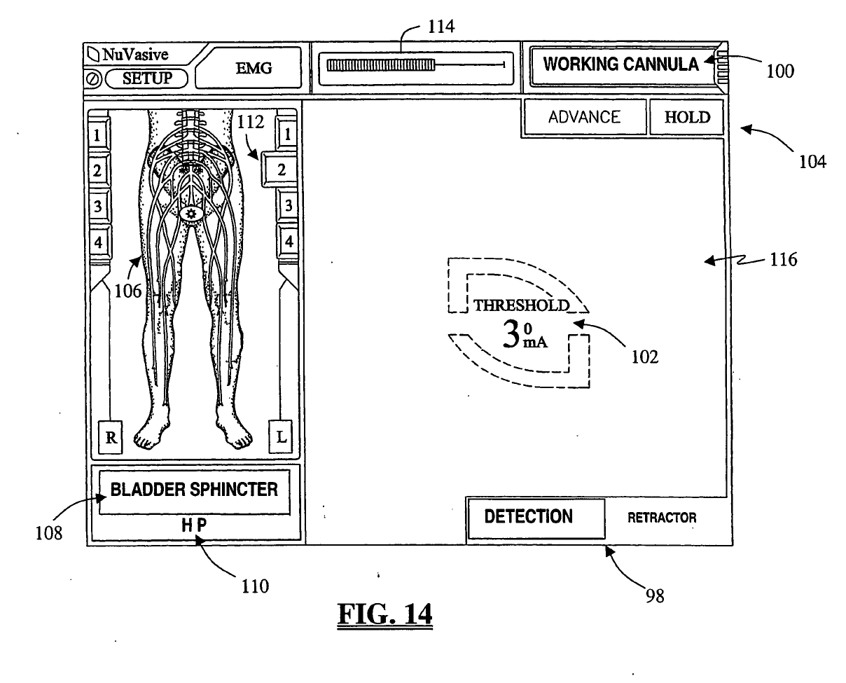

FIGS. 12A-12D illustrate, by way of example only, a threshold-hunting algorithm that employs a series of monopolar electrical stimulations to determine the stimulation current threshold Iₜₕᵣₑₛₕ for each EMG channel in range. The nerve is stimulated using current pulses with amplitude of Iₛₜᵢₘ. The muscle groups respond with an evoked potential that has a peak-to-peak voltage of Vₚₚ. The object of this algorithm is to quickly find Iₜₕᵣₑₛₕ, which once again, is the minimum Iₛₜᵢₘ that results in a Vₚₚ that is greater than a known threshold voltage Vₜₕᵣₑₛₕ. The value of Iₛₜᵢₘ is adjusted by a bracketing method as follows. The first bracket is 0.2 mA and 0.3 mA. If the Vₚₚ corresponding to both of these stimulation currents is lower than Vₜₕᵣₑₛₕ, then the bracket size is doubled to 0.2 mA and 0.4 mA. This exponential doubling of the bracket size continues until the upper end of the bracket results in a Vₚₚ that is above Vₜₕᵣₑₛₕ. The size of the brackets is then reduced by a bisection method. A current stimulation value at the midpoint of the bracket is used and if this results in a Vₚₚ that is above Vₜₕᵣₑₛₕ, then the lower half becomes the new bracket. Likewise, if the midpoint Vₚₚ is below Vₜₕᵣₑₛₕ then the upper half becomes the new bracket. This bisection method is used until the bracket size has been reduced to Iₜₕᵣₑₛₕ mA. I_{Thresh} is the value of Iₛₜᵢₘ that is the higher end of the bracket.

As discussed above, a pressure sensor rather than EMG electrodes may be employed to monitor the muscle response of the bladder sphincter. The basic technique behind the surgical system's 10 threshold hunting method remains the same, that is, to identify the minimum stimulation current Iₛₜᵢₘ capable of resulting in a predetermined muscle response (ie. Iₜₕᵣₑₛₕ). Muscle response is measured in terms of a pressure increase ΔP which may be substituted for Vₚₚ in the Iₜₕᵣₑₛₕ calculation. Thus, Iₜₕᵣₑₛₕ becomes the minimum Iₛₜᵢₘ that evokes a ΔP muscle response greater than a know threshold pressure increase (Pₜₕᵣₑₛₕ). The threshold hunting algorithm for quickly finding Iₜₕᵣₑₛₕ, shown in FIG. 12A-12D, may be employed by the system 10 by completing the bracketing and bisection steps discussed above, again substituting ΔP and Pₜₕᵣₑₛₕ for Vₚₚ and Vₜₕᵣₑₛₕ.

The threshold hunting will support three states: bracketing, bisection, and monitoring. A stimulation current bracket is a range of stimulation currents that bracket the stimulation current threshold I_{Thresh}. The upper and/or lower boundaries of a bracket may be indeterminate. The width of a bracket is the upper boundary value minus the lower boundary value. If the stimulation current threshold I_{Thresh} of a channel exceeds the maximum stimulation current, that threshold is considered out-of-range. During the bracketing state, threshold hunting will employ the method below to select stimulation currents and identify stimulation current brackets for each EMG channel in range.

The method for finding the minimum stimulation current uses the methods of bracketing and bisection. The “root” is identified for a function that has the value −1 for stimulation currents that do not evoke adequate response; the function has the value +1 for stimulation currents that evoke a response. The root occurs when the function jumps from −1 to +1 as stimulation current is increased: the function never has the value of precisely zero. The root will not be known precisely, but only with some level of accuracy. The root is found by identifying a range that must contain the root. The upper bound of this range is the lowest stimulation current I_{Thresh} where the function returns the value +1 (i.e. the minimum stimulation current that evokes response).

The nerve proximity function begins by adjusting the stimulation current from on the surgical instrument until the root is bracketed (FIG. 12B). The initial bracketing range may be provided in any number of suitable ranges. In one embodiment, the initial bracketing range is 0.2 to 0.3 mA. If the upper stimulation current does not evoke a response, the upper end of the range should be increased. The range scale factor is 2. The stimulation current should never be increased by more than 10 mA in one iteration. The stimulation current should never exceed the programmed maximum stimulation current. For each stimulation, the algorithm will examine the response of each active channel to determine whether it falls within that bracket. Once the stimulation current threshold of each channel has been bracketed, the algorithm transitions to the bisection state.

During the bisection state (FIG. 12C) threshold hunting will employ the method described below to select stimulation currents and narrow the bracket to a width of 0.1 mA for each channel with an in-range threshold. After the minimum stimulation current has been bracketed (FIG. 12B), the range containing the root is refined until the root is known with a specified accuracy. The bisection method is used to refine the range containing the root. In one embodiment, the root should be found to a precision of 0.1 mA. During the bisection method, the stimulation current at the midpoint of the bracket is used. If the stimulation evokes a response, the bracket shrinks to the lower half of the previous range. If the stimulation fails to evoke a response, the bracket shrinks to the upper half of the previous range. The algorithm is locked on the electrode position when the response threshold is bracketed by stimulation currents separated by 0.1 mA. The process is repeated for each of the active channels until all thresholds are precisely known. At that time, the algorithm enters the monitoring state.

During the monitoring state (FIG. 12D), threshold hunting will employ the method described below to select stimulation currents and identify whether stimulation current thresholds are changing. In the monitoring state, the stimulation current level is decremented or incremented by 0.1 mA, depending on the response of a specific channel. If the threshold has not changed then the lower end of the bracket should not evoke a response, while the upper end of the bracket should. If either of these conditions fail, the bracket is adjusted accordingly. The process is repeated for each of the active channels to continue to assure that each threshold is bracketed. If stimulations fail to evoke the expected response three times in a row, then the algorithm transitions back to the bracketing state in order to reestablish the bracket.

When it is necessary to determine the stimulation current thresholds (Iₜₕᵣₑₛₕ) for more than one channel, such as by way of example only, when monitoring is conducted on the bladder sphincter and the anal sphincter simultaneously, they will be obtained by time-multiplexing the threshold-hunting algorithm as shown in FIG. 13. During the bracketing state, the algorithm will start with a stimulation current bracket of 0.2 mA and increase the size of the bracket exponentially. With each bracket, the algorithm will measure the Vₚₚ of all channels to determine which bracket they fall into. After this pass, the algorithm will know which exponential bracket contains the Iₜₕᵣₑₛₕ for each channel. Next, during the bisection state, the algorithm will start with the lowest exponential bracket that contains an Iₜₕᵣₑₛₕ and bisect it until Iₜₕᵣₑₛₕ is found within 0.1 mA. If there are more than one Iₜₕᵣₑₛₕ within an exponential bracket, they will be separated out during the bisection process, and the one with the lowest value will be found first. During the monitoring state, the algorithm will monitor the upper and lower boundaries of the brackets for each Iₜₕᵣₑₛₕ, starting with the lowest. If the Iₜₕᵣₑₛₕ for one or more channels is not found in it's bracket, then the algorithm goes back to the bracketing state to re-establish the bracket for those channels.

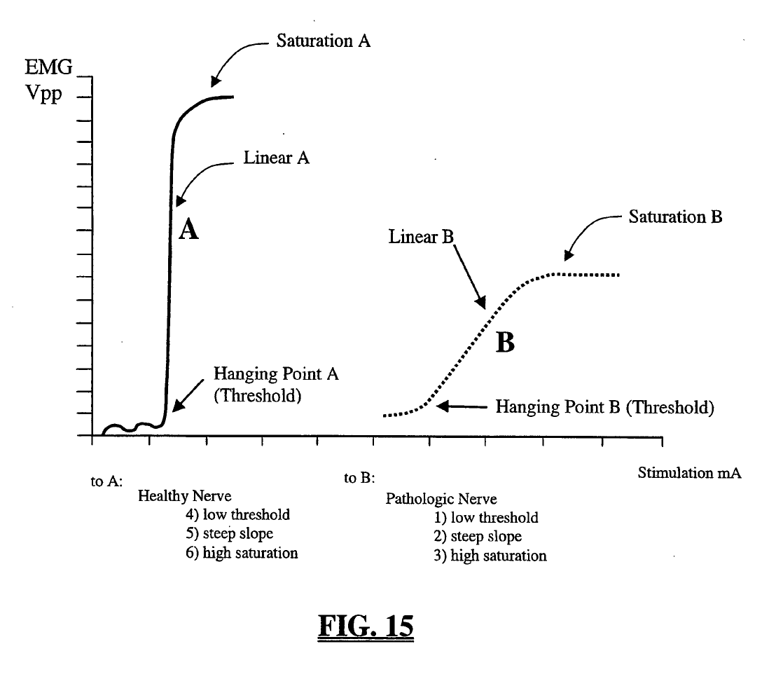

In one embodiment, the value of Iₜₕᵣₑₛₕ is displayed to the surgeon along with a color code so that the surgeon may easily comprehend the situation and avoid neurological impairment to the patient. The colors Red, Yellow, and Green are preferably displayed to indicate to the surgeon the level of safety determined by the system 10. Red is used to indicate an Iₜₕᵣₑₛₕ level below a predetermined unsafe level. Yellow indicates an Iₜₕᵣₑₛₕ that falls in between predetermined safe and unsafe levels. Green represents an Iₜₕᵣₑₛₕ within the range predetermined as safe. The actual Iₜₕᵣₑₛₕ value is generally only displayed when it falls in the Red (unsafe) range. However, the surgeon may select to have the actual Iₜₕᵣₑₛₕ value displayed for all ranges. FIG. 14, shown by way of example only, depicts an exemplary screen display of the present invention. The information on the screen may include, but is not necessarily limited to, the function 98 (in this case “Detection”), the instrument in use 100 (in this case “Working Cannula”), a display area 116 for showing procedure specific information such as the threshold stimulation current 102, instructions for the user 104 (in this case “Advance” or “Hold”), a graphical representation of the patient/spine 106, an indication of the moyotome or myotomes being monitored 108, an indication of the nerve group being monitored 110 (in this case the Hypogastric Plexus (HP)), channel tabs 112 indicating the selected channel when appropriate (ie.. when monitoring both the anal sphincter and the bladder sphincter together), and a stimulation bar 114 indicating the stimulation current being applied to the electrodes. In one embodiment, a green display corresponds to a stimulation threshold range of 10 milliamps (mA) or greater, a yellow display denotes a stimulation threshold range of 5-9 mA, and a red display denotes a stimulation threshold range of 4 mA or below. Additionally, at the option of the user, actual waveforms may displayed in conjunction with the corresponding stimulation result. insertion and advancement of the access instruments 30-34, 64 should be performed at a rate sufficiently slow to allow the surgical system 10 to provide real-time indication of the presence of the Hypogastric Plexus which may lie in the path of the tip. To facilitate this, the threshold current Iₜₕᵣₑₛₕ may be displayed such that it will indicate when the computation is finished and the data is accurate. For example, when the detection information is up to date such that the instrument is now ready to be advanced by the surgeon, it is contemplated to have the color display show up as saturated to communicate this fact to the surgeon. During advancement of the instrument, if a channel's color range changes from green to yellow, advancement should proceed more slowly, with careful observation of the detection level. If the channel color stays yellow or turns green after further advancement, it is a possible indication that the instrument tip has passed, and is moving farther away from the nerve. If after further advancement, however, the channel color turns red, then it is a possible indication that the instrument tip has moved closer to the nerve. At this point the display will show the value of the stimulation current threshold in mA. Further advancement should be attempted only with extreme caution, while observing the threshold values, and only if the clinician deems it safe. If the clinician decides to advance the instrument tip further, an increase in threshold value (e.g. from 3 mA to 4 mA) may indicate the Instrument tip has safely passed the nerve. It may also be an indication that the instrument tip has encountered and is compressing the nerve. The latter may be detected by listening for sporadic outbursts, or “pops”, of nerve activity on the free running EMG audio output (as mentioned above). If, upon further advancement of the instrument, the alarm level decreases (e.g., from 4 mA to 3 mA), then it is very likely that the instrument tip is extremely close to the Hypogastric Plexus, and to avoid neural damage, extreme caution should be exercised during further manipulation of the instrument. Under such circumstances, the decision to withdraw, reposition, or otherwise maneuver the instrument is at the sole discretion of the surgeon based upon available information and experience. Further radiographic imaging may be deemed appropriate to establish the best course of action.

As noted above, the surgical system 10 accomplishes neural pathology monitoring by electrically stimulating the hypogastric plexus via one or more stimulation electrodes at the distal end of the nerve root retractor 60 while monitoring the neuromuscular responses of the muscle group innervated by the particular nerve. FIG. 15 shows the differences between a healthy nerve (A) and a pathologic or unhealthy nerve (B). The inventors have found through experimentation that information regarding nerve pathology (or “health” or “status”) can be extracted from the recruitment curves generated according to the present invention (see, e.g., discussion accompanying FIGS. 8-11). In particular, it has been found that a healthy nerve or nerve bundle will produce a recruitment curve having a generally low threshold or “hanging point” (in terms of both the y-axis or Vₚₚ value and the x-axis or I_{Stim} value), a linear region having a relatively steep slope, and a relatively high saturation region (similar to those shown on recruitment curve “A” in FIG. 15). On the contrary, a nerve or nerve bundle that is unhealthy or whose function is otherwise compromised or impaired (such as being impinged by spinal structures or by prolonged retraction) will produce recruitment curve having a generally higher threshold (again, in terms of both the y-axis or Vₚₚ value and the x-axis or I_{Stim} value), a linear region of reduced slope, and a relatively low saturation region (similar to those shown on recruitment curve “B” in FIG. 15). By recognizing these characteristics, one can monitor nerve root being retracted during a procedure to determine if its pathology or health is affected (i.e. negatively) by such retraction. Moreover, one can monitor a nerve root that has already been deemed pathologic or unhealthy before the procedure (such as may be caused by being impinged by bony structures or a bulging annulus) to determine if its pathology or health is affected (i.e. positively) by the procedure.

In a preferred embodiment nerve pathology is monitored via the Nerve Retractor function specifically by determining a baseline stimulation threshold with direct contact between the nerve retractor 60 and the nerve but prior to retraction. Subsequently, additional stimulation thresholds are determined during retraction and they are compared to the baseline threshold. Significant changes in the stimulation threshold may indicate potential trauma to the nerve caused by the retraction. The information regarding nerve pathology may then be conveyed to the user screen display.

The surgical system 10 and related methods have been described above according to one embodiment of the present invention. It will be readily appreciated that various modifications may be undertaken, or certain steps or algorithms omitted or substituted, without departing from the scope of the present invention. By way of example only, certain of these alternate embodiments or methods will be described. In one alternative, rather than identifying the stimulation current threshold (I_{Thresh}) based on a predetermined V_{Thresh} (such as described above), other means may be used within the scope of the present invention to determine I_{Thresh}, such as by way of example, linear regression. Additionally, the nerve pathology monitoring function described above may be employed for the purpose of monitoring the change, if any, in peripheral nerves during the course of the procedure. This may be accomplished by positioning additional stimulation electrodes anywhere on a surgical accessory that is likely to come in contact with a peripheral nerve during a surgical procedure. Recruitment curves can be generated and assessed in the same fashion described above.

Moreover, although described with reference to the surgical system 10, it will be appreciated as within the scope of the invention to perform nerve testing for an anterior approach as described herein with any number of different neurophysiology based testing, including but not limited to the “NIM SPINE” testing system offered by Medtronic Sofamor Danek, Inc.

While this invention has been described in terms of a best mode for achieving this invention's objectives, it will be appreciated by those skilled in the art that variations may be accomplished in view of these teachings without deviating from the spirit or scope of the present invention. For example, the present invention may be implemented using any combination of computer programming software, firmware or hardware. As a preparatory step to practicing the invention or constructing an apparatus according to the invention, the computer programming code (whether software or firmware) according to the invention will typically be stored in one or more machine readable storage mediums such as fixed (hard) drives, diskettes, optical disks, magnetic tape, semiconductor memories such as ROMs, PROMs, etc., thereby making an article of manufacture in accordance with the invention. The article of manufacture containing the computer programming code is used by either executing the code directly from the storage device, by copying the code from the storage device into another storage device such as a hard disk, RAM, etc. or by transmitting the code on a network for remote execution. As can be envisioned by one of skill in the art, many different combinations of the above may be used and accordingly the present invention is not limited by the specified scope.

1. A system for conducting nerve testing during surgical procedures employing an anterior approach to the lumbar region, comprising:
a surgical accessory capable of delivering an electrical stimulation signal to a nerve lying anterior to the spine;
a sensor configured to detect neuromuscular responses evoked by the stimulation signal; and
a control unit communicably linked to the stimulation accessory and the sensor.

2. The system of claim 1 and further, wherein the nerve testing is conducted during surgical procedures including at least one of total disc replacement, nucleus replacement, and interbody fusion.

3. The system of claim 1 and further, wherein at least one of the surgical accessory and sensor are adapted for use in at least one of a trans-peritoneal approach, retroperitoneal approach, and a minimally invasive laparoscopic approach.

4. The system of claim 1 and further, wherein the control unit is configured for at least one of (a) directing emission of the stimulation signal from the surgical accessory, (b) receiving and characterizing the neuromuscular response detected by the sensor, and (c) identifying a relationship between the stimulation signal and the neuromuscular response to complete the nerve test.

5. The system of claim 4 and further, wherein the sensor is configured to detect at least one of an EMG voltage output and pressure change and wherein the neuromuscular response is characterized by the magnitude of at least one of the voltage output and pressure change.

6. The system of claim 5 and further, wherein the magnitude of the EMG voltage output is characterized by a peak-to-peak amplitude.

7. The system of claim 5 and further, wherein the relationship identified is the threshold stimulation current necessary to evoke a threshold neuromuscular response, the threshold neuromuscular response being defined by a predetermined magnitude.

8. The system of claim 7 and further, wherein the nerve testing conducted includes at least one of nerve detection during anterior surgical access and pathology monitoring during nerve retraction.

9. The system of claim 8 and further, wherein the nerve is the hypogastric plexus.

10. The system of claim 9 and further, wherein the targeted muscle includes at least one of the bladder sphincter and the anal sphincter.

11. The system of claim 10 and further, wherein the sensor is coupled to a urinary catheter for deployment to the bladder sphincter.

12. The system of claim 11 and further, wherein the sensor contacts the bladder sphincter when the urinary catheter is inserted into the bladder.

13. The system of claim 12 and further, wherein the sensor is an EMG electrode having a generally annular shape for positioning around the exterior surface of the catheter.

14. The system of claim 13 and further, wherein the EMG electrode is fixed in position on the urinary catheter using at least one of a biocompatible adhesive, crimping, and an interference fit.

15. The system of claim 12 and further, wherein the sensor is a pressure sensing microchip and the closing or opening of the bladder sphincter creates a detectable pressure change.

16. The system of claim 12 and further, wherein the sensor is fully integrated into the urinary catheter.

17. The system of claim 10 and further, wherein the sensor is coupled to an anal probe for deployment to the anal sphincter.

18. The system of claim 17 and further, wherein the sensor contacts the anal sphincter when the probe is positioned within the rectum.

19. The system of claim 10 and further, wherein the sensor is an EMG electrode.

20. The system of claim 19 and further, wherein one or more electrodes are placed on the surface around the anal sphincter.

21. The system of claim 8 and further, wherein the nerve test conducted is nerve detection during surgical access and the stimulation accessory includes at least one of fingertip electrode, a K-wire, dilating cannula, a working cannula, and a tissue retraction assembly.

22. The system of claim 21 and further, wherein the fingertip electrode comprises a stimulation electrode positioned on the fingertip region of a surgical glove.

23. The system of claim 8 and further, wherein the nerve test conducted is nerve pathology monitoring and the stimulation accessory is a nerve retractor.

24. The system of claim 8 and further, wherein the determined threshold stimulation current provides an indication of the proximity of the stimulation accessory to the nerve during surgical access and of nerve health during nerve retraction.

25. The system of claim 24 and further, wherein the control unit executes a hunting algorithm to determine the threshold stimulation current.

26. The system of claim 25 and further, wherein the system further includes a display coupled to the control unit and the control unit is configured to display at least one of a color and a numerical value relating to the determined threshold stimulation current.

27. The system of claim 25 and further, wherein the control unit is configured to employ an audible sound relating to the determined threshold stimulation current.

28. The system of claim 26 and further, wherein the display further includes a graphical user interface (GUI) configured to receive instructions from the user.

29. A method for conducting nerve testing during surgical procedures employing an anterior approach to the spine, comprising the steps of:
(a) delivering an electrical stimulation signal to a nerve lying anterior to the spine; and
(b) detecting neuromuscular responses evoked by the stimulation signal

30. The method of claim 29 and further, wherein the nerve testing is conducted during surgical procedures including at least one of total disc replacement, nucleus replacement, and interbody fusion.

31. The method of claim 29 and further, wherein the nerve testing is conducted during anterior surgical approaches including at least one of a trans-peritoneal approach, retroperitoneal approach, and a minimally invasive laparoscopic approach.

32. The method of claim 29 and further, wherein the control unit is configured for at least one of (a) communicating with a surgical accessory to direct the emission of the stimulation signal from the stimulation accessory, (b) communicating with a sensor configured to detect neuromuscular responses to receive and characterize the neuromuscular responses detected by the sensor, and (c) identifying a relationship between the stimulation signal and the neuromuscular response to complete the nerve test.

33. The method of claim 32 and further, wherein the sensor is configured to detect at least one of an EMG voltage output and pressure change and wherein the neuromuscular response is characterized by the magnitude of at least one of the voltage output and pressure change.

34. The method of claim 33 and further, wherein the magnitude of the EMG voltage output is characterized by a peak-to-peak amplitude.

35. The method of claim 33 and further, wherein the relationship identified is the threshold stimulation current necessary to evoke a threshold neuromuscular response, the threshold neuromuscular response being defined by a predetermined magnitude.

36. The method of claim 35 and further, wherein the nerve testing conducted includes at least one of nerve detection during anterior surgical access and pathology monitoring during nerve retraction.

37. The method of claim 36 and further, wherein the nerve is the hypogastric plexus.

38. The method of claim 37 and further, wherein the targeted muscle includes at least one of the bladder sphincter and the anal sphincter.

39. The method of claim 38 and further, wherein the sensor is coupled to a urinary catheter for deployment to the bladder sphincter.

40. The method of claim 39 and further, wherein the sensor contacts the bladder sphincter when the urinary catheter is inserted into the bladder.

41. The method of claim 40 and further, wherein the sensor is an EMG electrode having a generally annular shape for positioning around the exterior surface of the catheter.

42. The method of claim 41 and further, wherein the EMG electrode is fixed in position on the urinary catheter using at least one of a biocompatible adhesive, crimping, and an interference fit.

43. The method of claim 40 and further, wherein the sensor is a pressure sensing microchip and the closing or opening of the bladder sphincter creates a detectable pressure change.

44. The method of claim 40 and further, wherein the sensor is fully integrated into the urinary catheter.

45. The method of claim 38 and further, wherein the sensor is coupled to an anal probe for deployment to the anal sphincter.

46. The method of claim 45 and further, wherein the sensor contacts the anal sphincter when the probe is positioned within the rectum.

47. The method of claim 38 and further, wherein the sensor is an EMG electrode.

48. The method of claim 47 and further, wherein one or more electrodes are placed on the surface around the anal sphincter.

49. The method of claim 36 and further, wherein the nerve test conducted is nerve detection during surgical access and the stimulation accessory includes at least one of fingertip electrode, a K-wire, dilating cannula, a working cannula, and a tissue retraction assembly.

50. The method of claim 49 and further, wherein the fingertip electrode comprises a stimulation electrode positioned on the fingertip region of a surgical glove.

51. The method of claim 36 and further, wherein the nerve test conducted is nerve pathology monitoring and the stimulation accessory is a nerve retractor.

52. The method of claim 36 and further, wherein the determined threshold stimulation current provides an indication of the proximity of the stimulation accessory to the nerve during surgical access and of nerve health during nerve retraction.

53. The method of claim 52 and further, wherein the control unit executes a hunting algorithm to determine the threshold stimulation current.

54. The method of claim 53 and further including a display coupled to the control unit, and wherein the control unit is configured to display at least one of a color and a numerical value relating to the determined threshold stimulation current.

55. The method of claim 53 and further, wherein the control unit is configured to employ an audible sound relating to the determined threshold stimulation current.

56. The method of claim 54 and further, wherein the display further includes a graphical user interface (GUI) configured to receive instructions from the user.

57. A method for conducting nerve testing comprising the steps of:
(a) electrically stimulating the hypogastric plexus; and
(b) detecting a neuromuscular response from at least one of the bladder sphincter and the anal sphincter.
